(19) United States
(12) Patent Application Publication
(10) Pub. No.: US 2026/0294409 A1
(43) Pub. Date: Oct. 1, 2026

(54) SUPERELASTIC BIOPSY SHAFT AND STEERABLE NEEDLE

(71) Applicant: Auris Health, Inc., Santa Clara, CA (US)

(72) Inventors: Justin GILBERT, Santa Clara, CA (US); Akira UEDA, San Francisco, CA (US)

(73) Assignee: Auris Health, Inc., Santa Clara, CA (US)

(21) Appl. No.: 19/092,993

(22) Filed: Mar. 27, 2025

(51) Int. Cl. A61B 10/04; A61B 10/02

(52) U.S. Cl. CPC A61B 10/04; A61B 10/0233; A61B 2010/045

(57) ABSTRACT

A medical system includes a handle, an elongate shaft extending from the handle and defining a working channel, and a needle assembly extendable within the inner working channel and operable to translate distally and proximally relative to the elongate shaft. The needle assembly includes a sheath, and a needle arranged within the sheath and movable relative thereto. The needle has a body providing opposing distal and proximal ends and being made entirely of a superelastic material between the distal and proximal ends. The distal end of the needle extends through and past the handle.

### BACKGROUND

Endoscopy (e.g., bronchoscopy) may involve accessing and visualizing the inside of a patient's luminal network for diagnostic and/or therapeutic purposes. During a procedure a flexible tubular tool, known as an endoscope, may be inserted into the patient's body and a tool can be passed down through the endoscope to a tissue site identified for subsequent diagnosis and/or treatment. The endoscope can have an interior lumen (e.g., “working channel”) providing a pathway to the tissue site, and catheters and/or various medical tools can be inserted through the working channel to the tissue site.

### SUMMARY OF THE INVENTION

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to embodiments of the present disclosure, a medical system includes a handle, an elongate shaft extending from the handle and defining a working channel, and a needle assembly extendable within the inner working channel and operable to translate distally and proximally relative to the elongate shaft, the needle assembly including a sheath and a needle arranged within the sheath and moveable relative thereto, the needle having a body providing opposing distal and proximal ends and being made entirely of a superelastic material between the distal and proximal ends, wherein the distal end of the needle extends through and past the handle.

In accordance with other embodiments, a method of operating a medical system includes advancing an elongate shaft of the medical system into a patient cavity of a subject patient, the elongate shaft extending from a handle and defining a working channel, advancing a needle assembly within the inner working channel, the needle assembly including a sheath and a needle arranged within the sheath and movable relative thereto, the needle having a body providing opposing distal and proximal ends and being made entirely of a superelastic material between the distal and proximal ends, wherein the distal end of the needle extends through and past the handle, and advancing the needle out of the sheath and piercing a target tissue within the patient cavity.

In accordance with further embodiments, a needle assembly includes a sheath and a needle arranged within the sheath and movable relative thereto, the needle having a body providing opposing distal and proximal ends and being made entirely of a superelastic material between the distal and proximal ends, wherein the needle provides a distal region that is heat treated such that the superelastic material at the distal region transitions to an original shape memory state upon reaching a transformation temperature.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is an isometric view of an example medical system according to one or more embodiments.

FIGS. 2A and 2B are side and enlarged side views, respectively, of an example of the needle of FIG. 1.

FIGS. 3A and 3B are isometric views of an example of the needle assembly of FIG. 1.

FIGS. 4A and 4B illustrate the needle assembly of FIGS. 3A and 3B exhibiting superelastic properties in retracted and extended states, respectively.

FIGS. 5A and 5B are side views of another example needle assembly.

### DETAILED DESCRIPTION

Some medical procedures involve the manipulation of a tool positioned remotely from the operator, for example, positioned through a channel (e.g., trocars, catheters, endoscopes, etc.) inserted into the body of a patient. As one example of such a procedure, transbronchial needle aspiration (TBNA) can be used as a minimally invasive bronchoscopic technique for diagnosis and staging of bronchial diseases, including lung cancer. A TBNA technique can involve manipulating a biopsy needle through a flexible bronchoscope. For example, a physician can use chest scans to identify the location of a mass to be biopsied and to guide positioning of the bronchoscope within the patient's airways towards that mass. After the distal end of the bronchoscope working channel is positioned within the airways near the identified mass, an elongate, tubular jacket or “sheath” containing the biopsy needle can be advanced through the working channel to the sampling area. The target tissue can then be pierced by extending the needle out of the sheath, and aspiration can be applied to aid sample acquisition. Typically, sample acquisition involves moving the tube and the needle backward and forward relative to the bronchoscope to repeatedly puncture the tissue site with the needle (referred to as “dithering”). After sample acquisition, the needle can be retracted back into the sheath and withdrawn through the working channel.

Bronchoscopy techniques including TBNA can involve challenges accessing masses at the periphery of the lungs, particularly if such masses are relatively small (e.g., around 8 mm or smaller). Sampling masses at the periphery presents challenges in diagnosing and staging cancerous masses, particularly in early cancer stages, a timeframe during which such masses may be more easily treatable and may not have spread to other places in the patient's body. For example, a challenge in using a needle with a flexible bronchoscope is that the needle ought to be flexible enough to be maneuverable through the tortuous pathways of the bronchoscope to the target tissue site while also being rigid enough to deploy straight and allow penetration of the target tissue, and further that the needle preferably deploys in a straight trajectory over a distance sufficient to reach the lung periphery.

With respect to flexibility, bronchoscopes are increasingly minimizing the bend radiuses that they can achieve to allow the bronchoscopes to navigate through patient airways. With respect to rigidity, a bronchial wall or tumor tissue may present significant resistance to penetration by the needle. Some previous approaches use a short, rigid needle at the distal end of a plastic sheath to address these requirements so that the needle does not have to bend much as it travels through the bronchoscope. One example of an existing needle is a rigid needle approximately 7 mm or less in length. However, using this relatively short needle limits the extent to which the needle can be deployed straight—that is, the ability of the needle to be extended from the working channel with a proximal end still secured within the working channel. This can limit the range of the needle, preventing sampling of tissue at or near the periphery of the lungs. Further, the sheath cannot penetrate the tissue and thus causes trauma to patient tissue if it is jammed into the tissue, deflecting the tissue in order to allow for deeper penetration by the needle.

According to embodiments of the present disclosure, a needle assembly can include a “superelastic” needle that can be pre-formed into a shape (e.g., straight or curved) and capable of bending elastically through the tortuous pathways of a bronchoscope, returning to the pre-formed shape upon deployment from the bronchoscope, and deploying along the axis of the end of the bronchoscope (within a tolerated margin) with improved reach (e.g., in some embodiments, up to distances of 2 cm or more). The disclosed superelastic needle reduces scope tip deflection at the tip of the scope during the delivery of the needle through the working channel, which provides the desired accuracy in tissue sampling. In addition, the needles disclosed herein have aspiration/negative and/or positive pressure capabilities by having an open interior lumen. Thus, the disclosed needles can provide enhanced ability to sample tissue at the periphery of the lungs, for example, smaller lesions. Beneficially, this can allow a physician to diagnose and stage small, peripheral cancerous masses in earlier stages.

The disclosed systems and techniques provide advantages for bronchoscopic needle biopsies and other applications, including manipulation of other endoscopic, laparoscopic, and/or catheter-delivered tools. For example, a superelastic shaft similar to the disclosed needle can be provided for other types of medical tools, for example, augers, cytology brushes, and/or forceps. It will be appreciated that the needle dimensions described below could be similarly applicable to the dimensions of a superelastic shaft, and that the superelastic shaft may or may not be formed as a tubular shaft with an interior lumen. Thus, though the disclosed superelastic shafts are described in portions of the present disclosure below within the context of bronchoscopy biopsy needles, it should be understood that such shafts can also be used with other endoscopic tools and in other types of procedures in order to provide the disclosed benefits. For example, a superelastic medical tool as described herein can be used in other types of procedures including laparoscopy, gastrointestinal endoscopy, ureteroscopy, cardioscopy, and other procedures delivering tools through flexible and/or curved scopes, catheters, or tubes (collectively referred to as endoscopes, for simplicity of describing the various embodiments discussed herein).

As used herein, the term “superelastic” generally refers to a mechanical type of shape memory material in which an elastic (reversible) response to an applied stress is caused by a solid-solid phase transition. In some cases, superelastic effects are induced when a crystalline material in an austenite state is mechanically loaded up to a critical stress and within a specific temperature range above the martensitic transformation finishing temperature, at which point a phase transition to the martensite phase is induced. When so mechanically loaded, a superelastic material can deform reversibly to very high strains (for example, with Nitinol up to 10%) by the creation of such a stress-induced phase. When the load is removed, the martensite phase becomes unstable and the material undergoes the reverse deformation to regain its original shape. Further, no change in temperature is needed for the material to undergo this reverse deformation and recover this initial shape. Nitinol is a metal alloy of nickel and titanium that exhibits superelastic properties in a range of temperatures around room temperature. Other examples of superelastic materials include alloys of nickel and titanium with other elements (Ni—Ti—Fe, Ni—Ti—Cr, Ni—Ti—Cu—Cr), some polycrystalline ferrous alloys (e.g., Fe—Ni—Co—Al—Ta—B and Fe—Mn—Al—Ni), Cu—Al—Mn (CAM) alloys, and Cu—Zn—Sn alloys. It is to be appreciated that although the disclosed superelastic materials may be discussed herein with respect to implementation as a needle that is formed in, and reverts to, a straight shape, it will be appreciated that a medical tool formed from the disclosed superelastic materials can be formed to revert to a curved, angled, or otherwise non-straight shape in other implementations.

As used herein, “distal” refers to the end of the scope or tool positioned closest to the patient tissue site during use, and “proximal” refers to the end of the sheath or tool positioned closest to the operator (e.g., a physician or robotic control system). Stated differently, the relative positions of components of the jacket, needle, and/or the robotic system are described herein from the vantage point of the operator.

As used herein, the term “dithering” refers to a back and forth motion of a medical instrument such as a biopsy needle, for example during tissue sampling. The dithering movement of the needle can be independent of the movement of the needle's jacket such that the jacket of the needle remains relatively stationary during the dithering. Alternatively, “dithering” may refer to back and forth motion of the needle and the needle’s jacket together relative to the scope.

Robotic surgical systems can utilize endoscopic instruments to perform minimally invasive endoscopic procedures robotically. Some implementations of the present disclosure relate to surgical instruments and systems that include superelastic needles that can advantageously be used in robotically-guided (whether fully automated robotic systems or robotic systems that provide some level of assistance) medical procedures, and methods of performing a medical procedure under guidance of a robotic surgical system. In such systems, a robotic arm can be configured to control the extension, dithering, and/or retraction of needles as described herein. Drive signals for such actuation can be supplied by the robotic surgical system, for example, in response to user input via an input device and/or computer-controlled surgical processes. It will be appreciated, however, that the implementations of the present disclosure can equally be applied to manually operated surgical instruments and systems, without departing from the scope of the disclosure.

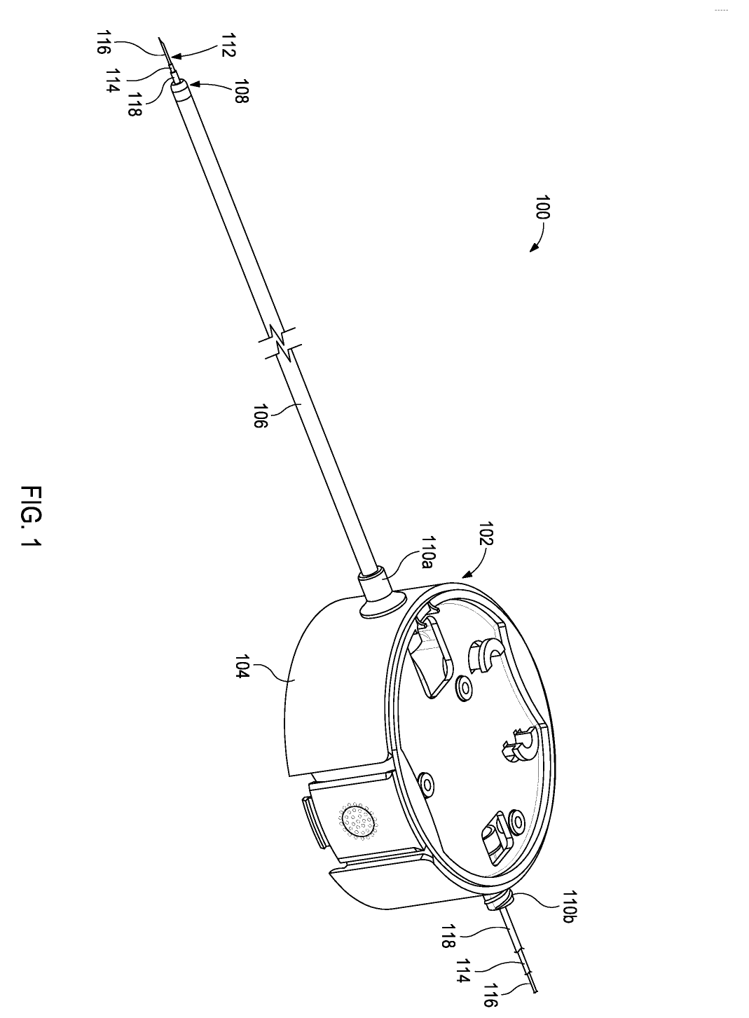

FIG. 1 is an isometric view of an example medical instrument 100 that may incorporate the principles of the present disclosure. As illustrated, the medical instrument 100 may include an endoscope 102 that includes a handle 104 (alternately referred to as a “drive housing” or an “instrument base”) and an elongate shaft 106 extending from the handle 104. The terms “endoscope” and “scope” may be used herein interchangeably and according to their broad and ordinary meanings, referring to any type of elongate (e.g., shaft-type) medical instrument having image generating, viewing, and/or capturing functionality and being configured to be introduced into any type of organ, cavity, lumen, chamber, or space of a body/subject. The term(s) can also include a catheter with or without vision (e.g., a sheath or equivalent). For example, the endoscope 102 can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, catheter (e.g., a steerable or non-steerable catheter), and so on. In some embodiments, the endoscope 102 defines a working channel through which additional tools/medical instruments, such as lithotripters, basketing devices, forceps, laser devices, imaging devices, catheters, needles, needle assemblies, etc., can be introduced into a treatment site.

In the illustrated embodiment, the handle 104 comprises a robotically controllable handle configured to be mounted to and manipulated (actuated) by a robotic arm (not shown). In other embodiments, however, the handle 104 may comprise a hand-held or manually-actuatable handle configured to be manually manipulated by a physician/user to manually control movement of the endoscope 104.

The shaft 106 extends from the handle 104 and terminates at a distal end 108. A proximal end (not shown) of the shaft 106 is located at or within the handle 104, and the shaft 106 extends from the handle 104 at a first or “distal” port 110a provided on a sidewall of the handle 104. In some applications, the shaft 106 accommodates wires and/or optical fibers to transfer signals to or from an optical assembly provided at the distal end 108. The optical assembly can include an imaging device, for example, such as an optical camera, which can be used to capture images of an internal anatomical space. The shaft 106 can further accommodate optical fibers to carry light from proximately-located light sources, such as light-emitting diodes, to the distal end 108. In such applications, the distal end 108 can include ports for light sources to illuminate the anatomical space when using the camera or imaging device.

At least a portion of the shaft 106 can be formed of a flexible material or construction to allow for articulation. Example materials or construction for the shaft 106 include, but are not limited to, plastics, rubbers, vertebrae links, metal or plastic braids/coils, or any combination thereof. In some applications, the shaft 106 includes reinforcement material (e.g., braided) to strengthen and/or facilitate flexibility of the shaft 106. For example, the shaft 106 can include braid reinforcement for hoop strength and or to prevent kinking of the shaft 106 when the shaft 106 is navigated within the anatomy of a patient. In some cases, the tip of the shaft 106 at the distal end 108 is formed of a different material than the rest of the shaft 106. For example, the tip can be implemented with a material that avoids degradation in certain contexts, such as catastrophic degradation. The tip can be implemented with stainless steel (or other types of steel), titanium, tungsten, and/or other materials (which may have relatively high melting points above a threshold) that can generally maintain its structure when laser beams inadvertently and/or occasionally contact the tip structure.

In some applications, the endoscope 102 comprises a steerable device, and in such applications, articulation of the shaft 106 may be manipulated via actuation (operation) of the handle 104. In other applications, however, the endoscope 102 may comprise a non-steerable device. In applications where the endoscope 102 is articulable or steerable, the shaft 106 can include or define one or more lumens (not visible) disposed lengthwise in the sidewall of the shaft 106. The lumens can be equidistantly spaced from each other and accommodate a corresponding one or more elongate movement members (not shown) extending from the handle 104 and slidably disposed within respective lumens. The elongate movement members can include one or more pull wires, cables, fibers, and/or flexible shafts, and can be made of any suitable or desirable material, such as metallic and non-metallic materials, including stainless steel, Kevlar, tungsten, carbon fiber, and the like. In some embodiments, the endoscope 102 is configured to exhibit nonlinear behavior in response to forces applied by the one or more elongate movement members. The nonlinear behavior may be based on stiffness and/or compressibility of the endoscope 102, as well as variability in slack or stiffness between different elongate movement members.

The elongate movement members can be attached/extend to the distal end 108, and at the proximal end the elongate movement members are coupled to corresponding components of the handle 104 (e.g., an input assembly) configured to control articulation of the shaft 106, such as by deflecting the distal end 108 of the shaft 106. The handle 104 can be operable to pull (and/or release tension of) the elongate movement members within the corresponding lumens to cause the distal end 108 to deflect from a longitudinal axis. In some embodiments, the endoscope 102 is configured to move in two directions based on manipulation of the elongate movement members (e.g., up/down or right/left), but could alternatively be configured to move in four directions based on manipulation of the elongate movement members (e.g., up/down and right/left). In yet other embodiments, the endoscope 102 can move in any direction by using a combination of four primary directions and four elongate movement members.

The medical instrument 100 further includes a needle assembly 112 capable of traversing an inner working channel defined by the shaft 106 and extending (advancing) out the distal end 108. The needle assembly 112 includes at least a sheath 114 and a needle 116 received within (extending through) the sheath 114. The needle 116 extends within the sheath 114 and is capable of advancing past and extending out a distal tip (end) of the sheath 114. In some embodiments, the needle assembly 112 may further include a catheter 118, and a combination of the sheath 114 and the needle 116 may be able to traverse an inner working channel of the catheter 118, while the catheter 118 may be able to traverse the inner working channel of the shaft 106. In such embodiments, the sheath 114 and the needle 116 may be capable of advancing past and extending out a distal tip of the catheter 118. In other embodiments, however, the catheter 118 may be omitted and the combination of the sheath 114 and the needle 116 may be configured to traverse the inner working channel of the shaft 106 of the endoscope 102.

In the illustrated embodiment, the needle assembly 112 extends through the handle 102 and exits the handle 102 at a second or “proximal” port 110b provided on a sidewall of the handle 104 and arranged angularly opposite the distal port 110a. In embodiments that include the catheter 118, the needle assembly 112 may extend to a second handle (not shown) and the catheter 118 may be operatively coupled to the second handle such that operation (actuation) of the second handle articulates (steers) the catheter 118. Accordingly, in such embodiments, and similar to the shaft 106, the catheter 118 may include one or more elongate movement members (not shown) that are actuatable (e.g., pulling or releasing) to control articulation of the shaft of the catheter 118.

The steerable configuration of the endoscope 102 and/or the catheter 118 allows the needle 116 to advance to and access hard-to-reach locations within a patient’s anatomy, such as the peripheral areas of a lung. When accessing the peripheral areas of the lung, the endoscope 102 (and/or the catheter 118) must navigate a tortuous path, which can impose high strain on the needle 116. In conventional needle assemblies, high strain can permanently deform (bend) the needle and the catheter, which, in some cases, can lead to device failure. Upon locating a desired location within the anatomy of a patient, when the needle is extended from the sheath, the user (e.g., surgeon, etc.) expects that the needle will extend out of the sheath coaxially aligned with the central axis of the endoscope or catheter. If the needle is bent by passing through a tortuous pathway, however, the needle trajectory will be unknown and extending the needle from the sheath or catheter will result in the needle extending in an arcuate or curved trajectory. This can cause prolonged surgery and the need for additional fluoroscopy to ensure the needle accurately punctures the target. In contrast, the straighter and more concentric the needle is with the working channel of the endoscope (or catheter), the more control and confidence the user has that the needle will puncture and track through desired tissue.

One way to help mitigate or prevent permanently bending the needle when exposed to a tortuous path and associated high strain is to make the needle out of a superelastic material, like Nitinol. Some conventional needles have a small portion (section) made of a superelastic material, which is used as the biopsy needle, and is joined to a cheaper stainless steel shaft that extends proximally to the handle. This results in a highly flexible region at the distal end of the needle, and a stiffer stainless steel shaft region that requires laser cuts for added flexibility. This also requires a bond location between the superelastic material and the steel shaft, which can lead to fracture and stiffness discontinuity at the bonded interface.

According to embodiments of the present disclosure, the whole of the needle 116 may be made entirely of a superelastic material (e.g., Nitinol), from distal to proximal ends of the needle 116. This eliminates the cheaper stainless steel shaft and the need for bonding to such shafts, and also eliminates the need to laser cut the steel shaft to increase its flexibility. Rather, the entire shaft of the needle 116, all the way back to the handle 104 (and further, in some applications), may be made of the superelastic material. Accordingly, in at least one embodiment, one or more of the needle 116, the sheath 114, and the catheter 118 may extend through and past the handle 104. Moreover, and as described in more detail below, the stiffness of the needle 116 at its distal end may be tuned by heat treating the material to become austenitic, and thereby converting a distal region of the needle 116 into a shape memory material. As a result, when the heat-treated distal region of the needle 116 warms to a predetermined transformation temperature (e.g., the temperature of the surrounding anatomy of a patient), it will assume its straight shape memory (e.g., original state or shape). This results in a robust shaft for the needle 116 that is both flexible and straight once reaching the transformation temperature.

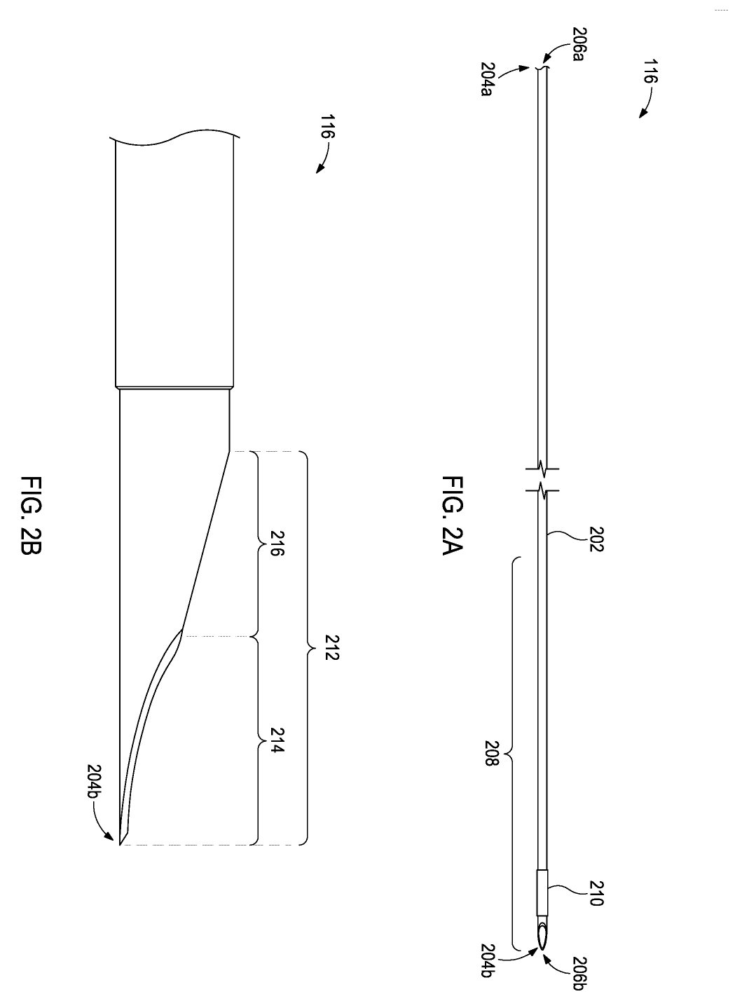

FIGS. 2A and 2B are side and enlarged side views, respectively, of an example of the needle 116 of FIG. 1, according to one or more embodiments. Referring first to FIG. 2A, as illustrated, the needle 116 includes an elongate body 202 formed as a thin-wall tube (e.g., a hypotube) and having a first or “proximal” end 204a and a second or “distal” end 204b opposite the proximal end 204a. The needle 116 is capable, in some embodiments, of delivering negative (or positive) pressure through an interior lumen extending between the proximal and distal ends 204a,b, and from a first aperture 206a defined at the proximal end 204b to a second aperture 206b at the distal end 204b. The first aperture 206a leads into the interior lumen, and aspiration or positive pressure can be applied through the first aperture 206a and delivered through the second aperture 206b of the needle 116. In other embodiments, however, the needle 116 may comprise a solid shaft, without departing from the scope of the disclosure.

In contrast to conventional needles, which are commonly bonded to a different material (e.g., stainless steel) to form (complete) the length of the needle shaft, the body 202 of the needle 116 is formed entirely of a superelastic material, from the proximal end 204a to the distal end 204b. The body 202 may extend through one or more proximally mounted drive housings or handles (e.g., the handle 102 of FIG. 1, and potentially a second handle arranged in series with the handle 102) included in the corresponding medical system (e.g., the medical instrument 100 of FIG. 1). In such embodiments, the proximal end 204a may be arranged proximal to the handle 102 and further proximal to a second handle (not shown), such as a needle handle, which is arranged in series with the handle 102. The needle handle may be configured to help control translation of the needle 116 through the inner working channel of the scope (e.g., the scope 106 of FIG. 1) or the inner working channel of the catheter (e.g., the catheter 118 of FIG. 1). Articulation (steering) of the catheter 118 and/or the scope 106 by the drive housing or handle will articulate (steer) the needle 116 contained therein, causing the needle 116 to bend or deflect without buckling or kinking along the length of the body 202 or, compared to some conventional needles, without buckling at a bonding site (e.g., when the needle 116 extends only to the handle 102).

In some applications, the superelastic material of the body 202 may be Nitinol, which is a nickel-titanium alloy with approximately equal parts nickel and titanium by atomic percent (e.g., a nickel to titanium ratio between 0.92 and 1.06). In such embodiments, the superelastic material can assume an interpenetrating simple cubic structure (referred to as the austenite phase) and can be set in this phase in the straight, tubular shape shown in FIG. 2A. When Nitinol in the austenite phase is subject to exterior forces in a temperature range from about −20° C. to +60° C., the Nitinol can undergo a phase transformation to the martensite phase as well as changing shape (e.g., to bend along its longitudinal axis as it travels through a bronchoscope). In the martensite phase, the crystal structure of the Nitinol shifts to a monoclinic structure, giving it the ability to undergo twinning deformation (e.g., the rearrangement of atomic planes without causing slip, or permanent deformation) without breaking atomic bonds. The Nitinol can reversibly undergo up to 10% strain in this manner. Upon release of the strain, the Nitinol automatically reverts back to the austenite phase and the original shape.

In other implementations, one or more other suitable superelastic materials capable of the austenite-martensite, solid-solid phase transformation can be used to form the needle 116. As described above, such materials include other alloys of nickel and titanium with other elements (Ni—Ti—Fe, Ni—Ti—Cr, Ni—Ti—Cu—Cr), some polycrystalline ferrous alloys (e.g., Fe—Ni—Co—Al—Ta—B and Fe—Mn—Al—Ni), Cu—Al—Mn (CAM) alloys, and Cu—Zn—Sn alloys. Thus, the needle 116 can be formed from superelastic materials having (1) a first solid crystal structure set to a straight, tubular shape in its austenite phase, (2) a second solid crystal structure that allows elastic deformation (e.g. bending) of the tubular shape up to a threshold strain percentage in its martensite phase, and (3) the capability to automatically revert from the martensite phase back to the austenite phase (and thus the original straight, tubular shape) upon release of the strain.

Nitinol, along with other superelastic materials, is a shape memory material. Shape memory is the ability of the material to undergo deformation at one temperature, stay in its deformed shape when the external force is removed, then recover its original, undeformed shape upon warming above its “transformation temperature”. In some embodiments, the needle 116 further includes a distal region 208 that is heat treated such that the material of the distal region 208 converts into a shape memory material capable of transitioning to its original state (i.e., shape) upon reaching a predetermined temperature (i.e., upon warming above its “transformation temperature”). The distal region 208 extends from the distal end 204b and may comprise (encompass), for example, about 1% to about 5% of the entire length of the body 202.

The heat treating process may be undertaken and otherwise tuned such that the transformation temperature of the distal region 208 may be, for example, the ambient temperature within the internal anatomy of a subject patient (e.g., 27° C – 40° C). Once reaching (e.g., warming to) the transformation temperature, the distal region 208 will revert back to its original shape memory state. It should be noted that other regions of the needle 116 may be heat treated, but the heat treatment of the distal region 208 will be different from any other region of the needle 116 such that a known original shape memory state of the distal region 208 can be achieved upon reaching the transformation temperature.

In at least one embodiment, the original shape memory state of the distal region 208 is straight. In such embodiments, when the needle 116 is advanced to a target site and the distal region 208 warms to the transformation temperature, the distal region 208 will become straight. This can be advantageous in scenarios where the needle 116 is required to bend as it passes through a tortuous pathway to reach the target site. While conventional needles could permanently bend after passing through a tortuous pathway, the heat treated distal region 208 is able to transition back to its straight, original shape memory state upon warming to the transformation temperature. In other embodiments, however, the original shape memory state of the distal region 208 may be arcuate or curved, without departing from the scope of the disclosure.

Some endoscopic procedures involve using fluoroscopy to assist with navigation of medical instruments through patient luminal networks. In fluoroscopy, a source of x-ray radiation is provided to pass an x-ray beam through patient tissue. This beam can be received by a screen positioned on the other side of the patient from the x-ray radiation source, and the resulting signal can be used to generate images (e.g., in grayscale or false color) to depict the internal structure of the patient. Radiopacity refers to the relative inability of electromagnetic radiation, particularly X-rays, to pass through a particular material. Materials that inhibit the passage of X-ray photons are referred to as radiopaque, while those that allow radiation to pass more freely are referred to as radiolucent or radio-transparent. The term “radiopaque,” as used herein, generally refers to the relatively opaque, white appearance of dense materials viewed in radiographic imaging, compared with the relatively darker appearance of less dense materials. Nitinol and some other superelastic materials can be radio-transparent or radiolucent, making these materials invisible or near-invisible when viewed in x-ray photographs or under fluoroscopy. This can make navigation of the needle 116 through a patient difficult when using fluoroscopy-based navigation systems.

Accordingly, in some embodiments, the needle 116 can optionally be provided with a radiopaque material 210 near the distal end 204b. In some examples, the radiopaque material 210 can be positioned around 3 mm from the distal end 204b of the needle 116. As described above, radiopaque materials are opaque to x-ray radiation and thus visible in x-ray photographs and under fluoroscopy. As such, although the material of the needle 116 may be radio-transparent, its navigation can be guided by observing the position of the radiopaque material 208. Providing the radiopaque material 208 near the distal end 204b provides an indication of how close the distal end 204b is to the target tissue site. In one example, this radiopaque material 208 can be formed as a thin gold band and secured around the exterior surface of the needle 116. Other implementations can use suitable radiopaque materials having high element content of elements with high atomic numbers, including tungsten, precious metals and alloys containing precious metals including chromium-nickel alloys (Cr—Ni), radiopaque ceramics, and radiopaque thermoplastics. In some embodiments the radiopaque material 208 may be secured to the interior of the needle 116.

Referring to FIG. 2B, the needle 116 is formed with a sharpened tip 212 at the distal end 204b. The sharpened tip 212 includes a lancet design where one zone 214 is ground flat at a first angle and a second zone 216 is ground flat at a different angle. The second zone 216 provides a linear slope to the end of the sharpened tip 212 that meets the full diameter of the needle 116.

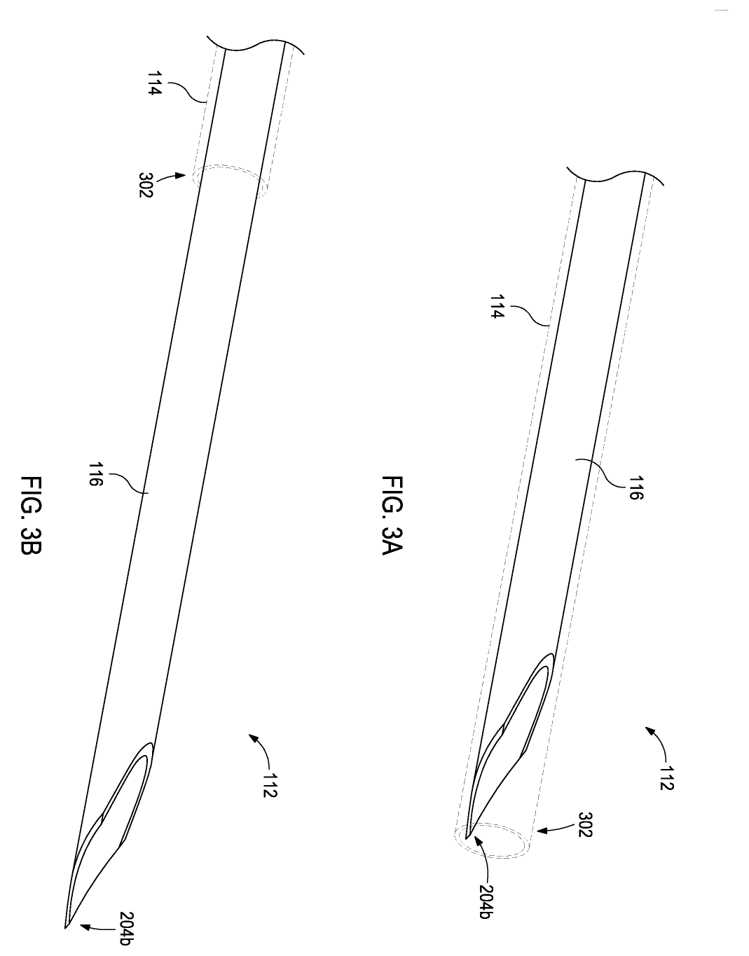

FIGS. 3A and 3B are isometric views of an example of the needle assembly 112 of FIG. 1, according to one or more embodiments. As illustrated, the needle assembly 112 includes the needle 116 and the sheath 114 (shown in phantom). The sheath 114 can be a polymer catheter or tube in some embodiments, and in other embodiments can be a steerable channel. The outer diameter of the sheath 114 can be selected to substantially match the interior diameter of the working channel of the catheter 118 (FIG. 1) or the shaft 106 (FIG. 1) of the endoscope 102 (FIG. 1) for secure centering of the needle 116 relative to the working channel.

FIG. 3A illustrates the needle assembly 112 in a retracted state, and FIG. 3B illustrates the needle assembly 112 in an extended state. In the retracted state, the distal end 204b of the needle 116 is positioned within the sheath, at or proximal to a distal end 302 of the sheath 114, and the sheath 114 essentially houses the entire needle 116. The proximal end (not shown) of the needle 116 may extend to a drive housing or “handle”, and beyond a proximal end (not shown) of the sheath 114 so that it can be moved relative to the sheath 114 between the retracted and extended states. In some embodiments, when the needle 116 is in a fully retracted state, the distal end 204b of the needle 116 may be positioned a certain distance within the sheath 114, for example 5 mm from the distal end 302 of the sheath 114.

In the extended state, the distal end 204b of the needle 116 is advanced distally beyond the distal end 302 of the sheath 114. The needle 116 can be driven distally from the retracted state to the extended state by manual or robotic movement of the needle 116, and can be driven proximally from the extended state to the retracted state by manual or robotic movement of the needle 116. In some embodiments the distal tip 204b of the needle 116 can extend 3 cm, or in the range of 2 cm to 4 cm, beyond the distal end 302 of the sheath 114 in the extended state, but could alternatively be configured to extend more than 4 cm beyond the distal end 302 of the sheath 114. The length of the needle 116 may be about 1 meter, but could alternatively be longer or shorter than 1 meter, without departing from the scope of the disclosure.

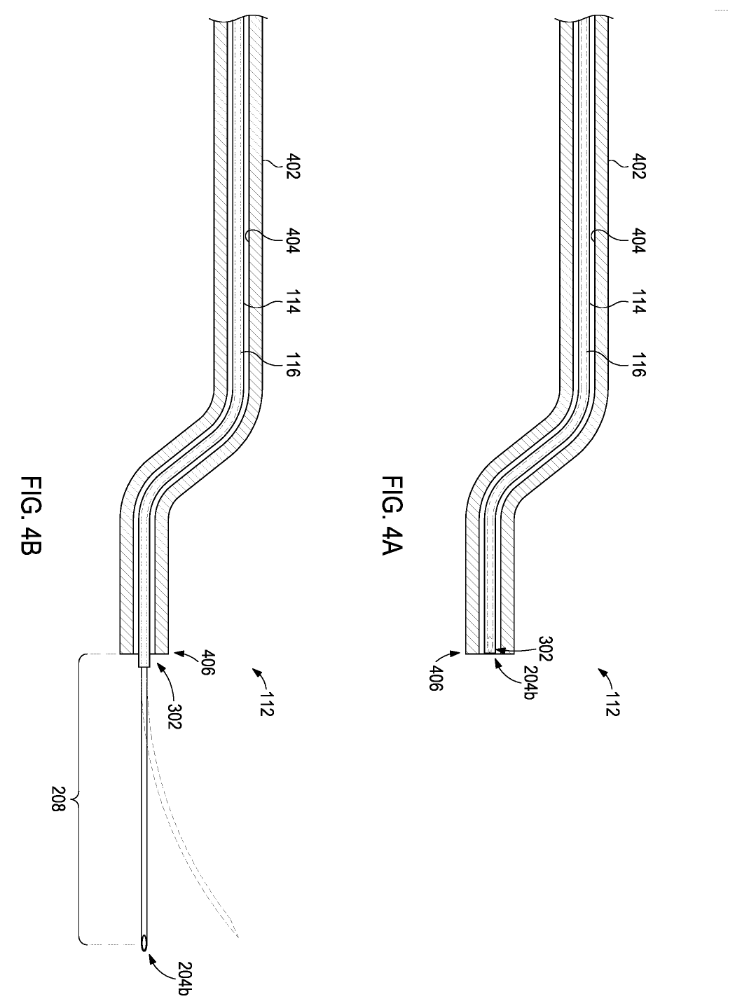

FIGS. 4A and 4B illustrate the needle assembly 112 of FIGS. 3A-3B exhibiting superelastic properties in retracted and extended states. In the illustrated embodiment, the needle assembly 112 is arranged within an elongate shaft 402, which may comprise the catheter 118 of FIG. 1, but could alternatively comprise the shaft 106 (FIG. 1) of the endoscope 102 (FIG. 1). The needle assembly 112 is positioned within a working channel 404 of the shaft 402, illustrated in cross-section to reveal the needle assembly 112.

In FIG. 4A, the needle 116 is shown in the retracted state and otherwise positioned with its distal tip 204b located within the sheath 114. The sheath 114 and the needle 116 can be advanced together through the working channel 404 of the shaft 402 until the distal end 302 of the sheath 114 and the distal end 204b of the needle 116 are positioned at (or near) a distal end 406 of the shaft 402 (e.g., the distal end 108 of the shaft 106 of FIG. 1). The sheath 114 may prevent the sharp point of the needle 116 at the distal end 204b from penetrating or binding on the inner wall of the working channel 404 as the needle assembly 112 is advanced within the working channel 404. In FIG. 4B, the needle 116 is transitioned to the extended state and otherwise advanced distally beyond the distal end 302 of the sheath 114.

As described above, the material properties of the needle 116 allow the needle 116 to undergo strain up to a certain threshold while deforming reversibly. As illustrated, the needle 116 is required to traverse at least two bends along its longitudinal axis as it is advanced through the working channel 404 of the shaft. The needle 116 is able to bend around a radius of curvature while deforming reversibly in the martensitic state. In one example, for instance, the needle 116 can elastically bend around a radius of 9-12 mm or greater, and in other examples the needle 116 can deform elastically around 180 degree bends. This deformation can occur repeatedly as the sheath 114 containing needle 116 is inserted through the working channel 404 of the shaft 402.

Moreover, as also described above, the distal region 208 (FIG. 4B) of the needle 116 may be heat treated such that the material of the distal region 208 is able to transition (revert) back to its original shape upon reaching a transformation temperature, which may be, for example, the ambient temperature within the internal anatomy of a subject patient (e.g., 27° C – 40° C). In the illustrated embodiment, as the needle 116 advances distally and out of the sheath 114, the distal region 208 enters a patient cavity and potentially comes into contact with the internal anatomy of the subject patient. Before warming to the ambient temperature within the internal anatomy (e.g., the transformation temperature), the distal region 208 of the needle 116 may be arcuate or curved, as shown in dashed lines. Once the temperature of the distal region 208 warms to the ambient temperature, however, the distal region 208 will transition to its original shape memory state, which, in this embodiment, is straight, and thus coaxial with a longitudinal axis of the shaft 402.

Beneficially, this automatic reverse deformation allows the needle 116 to traverse tight bends through the shaft 402 and still deploy substantially straight along the longitudinal axis extending out of the working channel 404. Such deployment increases accuracy with respect to sampling pre-identified target tissue regions.

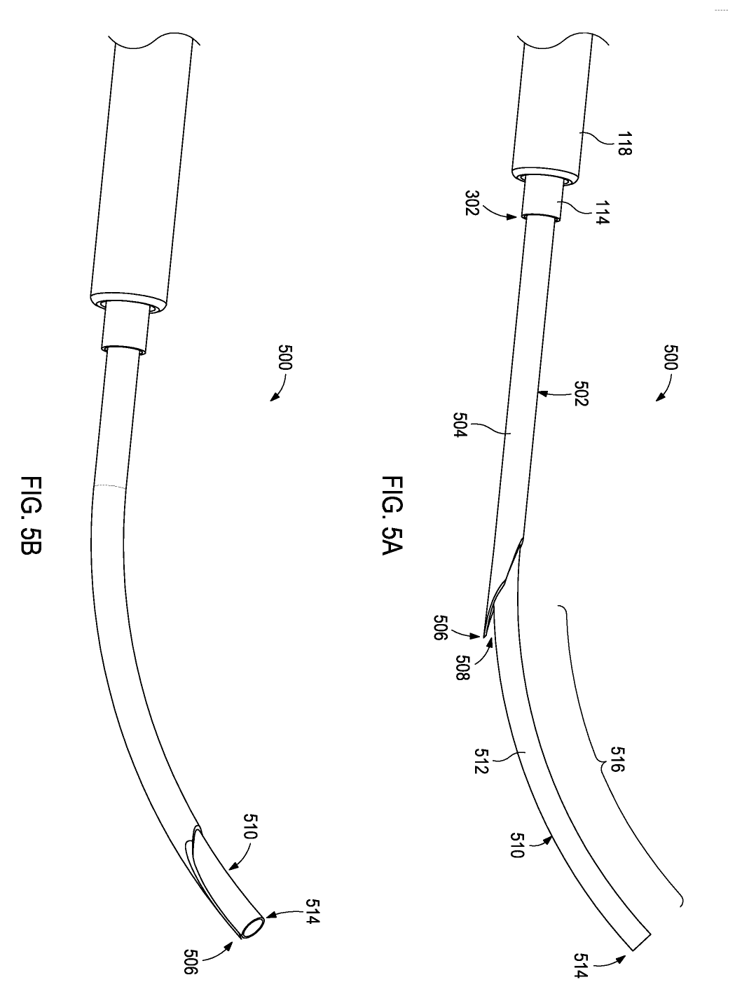

FIGS. 5A and 5B are side views of another example needle assembly 500, in accordance with various aspects of the present disclosure. The needle assembly 500 may be similar in some respects to the needle assembly 112 of FIGS. 1-4B and therefore may be best understood with reference thereto, where like reference numerals will correspond to similar components not described again in detail.

Referring first to FIG. 5A, as illustrated, the needle assembly 500 includes at least the sheath 114 and a needle 502 received within the sheath 114. The needle 502 extends within the sheath 114 and is capable of advancing past and extending out the distal end 302 of the sheath 114. In some embodiments, the needle 502 may be the same as the needle 116 of FIGS. 1-4B, but could alternatively be different from the needle 116. As illustrated, the needle 502 includes an elongate body 504 formed as a thin-wall tube (e.g., a hypotube) having a first or “proximal” end (not shown) and a second or “distal” end 506 opposite the proximal end. Similar to the needle 116, the needle 502 can be capable of delivering negative (or positive) pressure through an interior lumen extending between the proximal and distal end 506, and from a first aperture (not shown) defined at the proximal end to a second aperture 508 at the distal end 506.

In at least one embodiment, the needle 502 is made entirely of stainless steel and may be laser cut at various locations along its length to improve its flexibility. In other embodiments, and similar to the needle 116 of FIGS. 1-4B, the needle 502 may be made entirely of a superelastic material. In yet other embodiments, the needle 502 may comprise a combination of a superelastic material, such as nitinol, and a steel shaft bonded to the superelastic material.

In some embodiments, the needle assembly 500 further includes the catheter 118, and the sheath 114 and the needle 502 may be able to traverse the inner working channel of the catheter 118. In other embodiments, however, the catheter 118 may be omitted, and the sheath 114 and the needle 502 may be configured to traverse the inner working channel of the shaft 106 of the endoscope 102 (FIG. 1).

The needle assembly 500 further includes a stylet 510 translatable within the interior lumen of the needle 502. The stylet 510 may comprise an elongate shaft or wire used to help control stiffness to the needle 502, as well as to push an obtained sample out from the interior lumen of the needle 502. Unlike conventional stylets, however, the stylet 510 has a shaped distal region that guides or steers the needle 502 for puncture (penetration) in an intended trajectory. Depending on the location of the target tissue and the orientation of the anatomy, it may be quite difficult to angle the longitudinal axis of the catheter 118 (or the shaft 106 of the endoscope 102 of FIG. 1) to align perpendicular to a desired puncture location, and conventional needles may not be able to puncture the target tissue but instead deflect off the tissue wall. As described herein, the stylet 510 introduces the option to steer (guide) the needle 502 in a direction preferred by the physician to puncture, thus obtaining a best possible puncture angle.

As illustrated, the stylet 510 includes an elongate body 512 sized to be received within the interior lumen of the needle 502. The body 512 has a first or “proximal” end (not shown) and a second or “distal” end 514 opposite the proximal end. The stylet 510 is independently moveable relative to the needle 502 within the interior lumen. According to embodiments of the present disclosure, the entire length of the body 512 of the stylet 510 may be made entirely of a superelastic material (e.g., Nitinol). Moreover, the stylet 510 includes a distal region 516 at and extending from the distal end 514 that is heat treated such that the material of the distal region 516 is converted to a shape memory material capable of transitioning to its original state (i.e., shape) upon reaching a predetermined temperature (i.e., upon warming to or above its “transformation temperature”). In at least one embodiment, the distal region 516 may be heat treated such that the original shape memory state is curved or arcuate. Accordingly, once the temperature of the distal region 516 warms to the transformation temperature, the distal region 516 will transition to the curved or arcuate shape, as shown in FIG. 5A. Upon assuming the curved shape, the stylet 510 may be able to operate as a guidewire to guide the needle 502 in a curved trajectory and to a desired location.

FIG. 5A illustrates the needle assembly 500 in an extended configuration, and FIG. 5B illustrates the needle assembly 500 in a guiding configuration. In the extended configuration, the stylet 510 is advanced distally relative to the needle 502 such that the distal end 514 of the stylet 510 is extended beyond the distal end 506 of the needle 502. In the extended configuration, the distal region 516 of the stylet 510 becomes exposed to the internal anatomy of the subject patient, and upon warming to the transformation temperature (e.g., the ambient temperature within the internal anatomy; e.g., 27° C – 40° C), the distal region 516 will transition to its original shape memory state, which is curved or arcuate. In some applications, the stylet 510 may be advanced until engaging a target site, thus anchoring the stylet 510 within the subject at the target site, which acts as a reaction surface. In at least one embodiment, the stylet 510 may include a structure at its distal tip allowing the stylet 510 to be anchored into the tissue. In some embodiments, the stylet 510 may be used to axially support the needle 502 as the needle is advanced into the guiding configuration, thereby helping to prevent the needle 502 from buckling.

In the guiding configuration, as shown in FIG. 5B, the needle 502 is advanced distally relative to the stylet 510 and assumes the shape of the stylet 510. Once transitioned to the curved, original shape memory state, the distal region 516 will exhibit a flexibility that is less than the flexibility of the needle 502, thereby allowing the needle 502 to advance along the stylet 510 and assume the curved or arcuate shape of the stylet 510. In the guiding configuration, the needle 502 may be advanced until the distal end 506 of the needle 502 bypasses the distal end 514 of the stylet 510, thereby enabling the needle 502 to be steered into proper alignment with the target site, such as perpendicular or substantially perpendicular to the site of the biopsy. Once advanced, the needle 502 may be further deployed to sample the tissue.

The proximal end (not shown) of the needle 502 and the proximal end (not shown) of the stylet 510 may extend to a drive housing or “handle”, and beyond a proximal end (not shown) of the sheath 114. In other embodiments, the elongate body 504 of the needle 502 or the elongate body 512 of the stylet 510 may extend less than the full distance to the handle. Beneficially, the superelastic stylet 510 may induce desirable geometries of the needle 502 without the needle 502 needing to be superelastic. Accordingly, the needle 502 may be made of cheaper stainless steel, which may be sufficiently rigid when deployed from the sheath 114 to behave predictably.

Embodiments disclosed herein include:
A. A medical system including a handle, an elongate shaft extending from the handle and defining a working channel, and a needle assembly extendable within the inner working channel and operable to translate distally and proximally relative to the elongate shaft, the needle assembly including a sheath and a needle arranged within the sheath and movable relative thereto, the needle having a body providing opposing distal and proximal ends and being made entirely of a superelastic material between the distal and proximal ends, wherein the distal end of the needle extends through and past the handle.
B. A method of operating a medical system including advancing an elongate shaft of the medical system into a cavity of a subject, the elongate shaft extending from a handle and defining a working channel, advancing a needle assembly within the inner working channel, the needle assembly including a sheath and a needle arranged within the sheath and movable relative thereto, the needle having a body providing opposing distal and proximal ends and being made entirely of a superelastic material between the distal and proximal ends, wherein the distal end of the needle extends through and past the handle, and advancing the needle out of the sheath and piercing a target tissue within the cavity.
C. A needle assembly including a sheath and a needle arranged within the sheath and movable relative thereto, the needle having a body providing opposing distal and proximal ends and being made entirely of a superelastic material between the distal and proximal ends, wherein the needle provides a distal region that is heat treated such that the superelastic material at the distal region transitions to an original shape memory state upon reaching a transformation temperature.

Each of embodiments A, B, and C may have one or more of the following additional elements in any combination: Element 1: wherein the needle assembly further includes a catheter, and the sheath and the needle are received within the catheter and operable to advance past a distal end of the catheter. Element 2: wherein the needle provides a distal region that is heat treated such that the superelastic material at the distal region transitions to an original shape memory state upon warming to a transformation temperature. Element 3: wherein the distal region extends from the distal end of the needle and encompasses between about 1% and about 15% of a length of the body. Element 4: wherein the transformation temperature of the distal region comprises ambient temperature within internal anatomy of a subject. Element 5: wherein the original shape memory state of the distal region is straight. Element 6: wherein the original shape memory state of the distal region is curved. Element 7: wherein the needle assembly further includes a stylet translatable within an interior lumen of the needle, the stylet including a body providing opposing distal and proximal ends and a distal region made of a superelastic material that is heat treated such that the superelastic material at the distal region transitions to an original shape memory state upon warming to a transformation temperature. Element 8: wherein the original shape memory state of the distal region is curved. Element 9: wherein, when the distal region transitions to the original shape memory state, a flexibility of the distal region will be less than a flexibility of the needle such that advancing the needle over the distal region causes the needle to assume the shape of the stylet. Element 10: wherein the needle assembly further includes a catheter, and the sheath and the needle are received within the catheter, the method further including advancing the sheath and the needle within the catheter and past a distal end of the catheter. Element 11: wherein the needle provides a distal region that is heat treated such that the superelastic material at the distal region transitions to an original shape memory state upon reaching a transformation temperature, the method further including warming the distal region to the transformation temperature within the cavity, and thereby causing the distal region to transition to the original shape memory state. Element 12: wherein the needle assembly further includes a stylet arranged within an interior lumen of the needle, the stylet including a body providing opposing distal and proximal ends, and a distal region made of a superelastic material that is heat treated such that the superelastic material at the distal region transitions to an original shape memory state upon warming to a transformation temperature, the method further including advancing the stylet out of the interior lumen such that the distal region is exposed to the cavity, warming the distal region to the transformation temperature within the cavity, and thereby causing the distal region to transition to the original shape memory state, and advancing the needle over the distal region and thereby causing the needle to assume the shape of the stylet. Element 13: further including a catheter, wherein the sheath and the needle are received within the catheter and operable to advance past a distal end of the catheter. Element 14: wherein the superelastic material comprises Nitinol. Element 15: wherein the distal region extends from the distal end of the needle and encompasses between about 1% and about 15% of a length of the body. Element 16: wherein the transformation temperature of the distal region is between about 27° C and about 40° C. Element 17: wherein the original shape memory state of the distal region is straight.

By way of non-limiting example, exemplary combinations applicable to A, B, and C include: Element 2 with Element 3; Element 2 with Element 4; Element 2 with Element 5; Element 2 with Element 6; Element 7 with Element 8; Element 7 with Element 9.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the term “subject patient” can refer to a living human subject, but is equally applicable to a human cadaver, an animal, or a model used in testing, teaching, or training facilities. Accordingly, the term “subject patient” is used interchangeably herein with the general term “subject,” and “patient cavity” is used interchangeably herein with the general term “cavity,” thus encompassing all types of uses.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The headings provided herein are for convenience only and do not necessarily affect the scope or meaning of the disclosure. Although certain embodiments and examples are disclosed below, the subject matter extends beyond the specifically disclosed embodiments to other alternative embodiments and/or uses and to modifications and equivalents thereof. Thus, the scope of the claims that may arise here from is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

What is claimed is:

1. A medical system, comprising:
a handle;
an elongate shaft extending from the handle and defining a working channel; and
a needle assembly extendable within the inner working channel and operable to translate distally and proximally relative to the elongate shaft, the needle assembly including:
a sheath; and
a needle arranged within the sheath and movable relative thereto, the needle having a body providing opposing distal and proximal ends and being made entirely of a superelastic material between the distal and proximal ends,
wherein the distal end of the needle extends through and past the handle.

2. The medical system of claim 1, wherein the needle assembly further includes a catheter, and the sheath and the needle are received within the catheter and operable to advance past a distal end of the catheter.

3. The medical system of claim 1, wherein the needle provides a distal region that is heat treated such that the superelastic material at the distal region transitions to an original shape memory state upon warming to a transformation temperature.

4. The medical system of claim 3, wherein the distal region extends from the distal end of the needle and encompasses between about 1% and about 15% of a length of the body.

5. The medical system of claim 3, wherein the transformation temperature of the distal region comprises ambient temperature within internal anatomy of a subject.

6. The medical system of claim 3, wherein the original shape memory state of the distal region is straight.

7. The medical system of claim 3, wherein the original shape memory state of the distal region is curved.

8. The medical system of claim 1, wherein the needle assembly further includes a stylet translatable within an interior lumen of the needle, the stylet including:
a body providing opposing distal and proximal ends; and
a distal region made of a superelastic material that is heat treated such that the superelastic material at the distal region transitions to an original shape memory state upon warming to a transformation temperature.

9. The medical system of claim 8, wherein the original shape memory state of the distal region is curved.

10. The medical system of claim 8, wherein, when the distal region transitions to the original shape memory state, a flexibility of the distal region will be less than a flexibility of the needle such that advancing the needle over the distal region causes the needle to assume the shape of the stylet.

11. A method of operating a medical system, comprising:
advancing an elongate shaft of the medical system into a cavity of a subject, the elongate shaft extending from a handle and defining a working channel;
advancing a needle assembly within the inner working channel, the needle assembly including:
a sheath; and
a needle arranged within the sheath and movable relative thereto, the needle having a body providing opposing distal and proximal ends and being made entirely of a superelastic material between the distal and proximal ends, wherein the distal end of the needle extends through and past the handle; and
advancing the needle out of the sheath and piercing a target tissue within the cavity.

12. The method of claim 11, wherein the needle assembly further includes a catheter, and the sheath and the needle are received within the catheter, the method further comprising advancing the sheath and the needle within the catheter and past a distal end of the catheter.

13. The method of claim 11, wherein the needle provides a distal region that is heat treated such that the superelastic material at the distal region transitions to an original shape memory state upon reaching a transformation temperature, the method further comprising:
warming the distal region to the transformation temperature within the cavity, and thereby causing the distal region to transition to the original shape memory state.

14. The method of claim 11, wherein the needle assembly further includes a stylet arranged within an interior lumen of the needle, the stylet including a body providing opposing distal and proximal ends, and a distal region made of a superelastic material that is heat treated such that the superelastic material at the distal region transitions to an original shape memory state upon warming to a transformation temperature, the method further comprising:
advancing the stylet out of the interior lumen such that the distal region is exposed to the cavity;
warming the distal region to the transformation temperature within the cavity, and thereby causing the distal region to transition to the original shape memory state; and
advancing the needle over the distal region and thereby causing the needle to assume the shape of the stylet.

15. A needle assembly, comprising:
a sheath; and
a needle arranged within the sheath and movable relative thereto, the needle having a body providing opposing distal and proximal ends and being made entirely of a superelastic material between the distal and proximal ends,
wherein the needle provides a distal region that is heat treated such that the superelastic material at the distal region transitions to an original shape memory state upon reaching a transformation temperature.

16. The needle assembly of claim 15, further comprising a catheter, wherein the sheath and the needle are received within the catheter and operable to advance past a distal end of the catheter.

17. The needle assembly of claim 15, wherein the superelastic material comprises Nitinol.

18. The needle assembly of claim 15, wherein the distal region extends from the distal end of the needle and encompasses between about 1% and about 15% of a length of the body.

19. The needle assembly of claim 15, wherein the transformation temperature of the distal region is between about 27 °C and about 40 °C.

20. The needle assembly of claim 15, wherein the original shape memory state of the distal region is straight.
